(19) United States
(12) Patent Application Publication
(10) Pub. No.: US 2018/0370891 A1
(43) Pub. Date: Dec. 27, 2018

(54) CURCUMIN COMPOSITIONS FOR IMPROVEMENT IN CARDIOVASCULAR FLOW

(71) Applicant: OMNIACTIVE HEALTH TECHNOLOGIES LIMITED, Lower Parel, Mumbai (IN)

(72) Inventors: Jayant DESHPANDE, Charlottetown, Prince Edward Island (CA); Vijaya JUTURU, Morristown, NJ (US)

(73) Assignee: OMNIACTIVE HEALTH TECHNOLOGIES LIMITED, Lower Parel, Mumbai (IN)

(21) Appl. No.: 15/774,875

(22) PCT Filed: Nov. 9, 2016
(86) PCT No.: PCT/IB2016/056751

§ 371 Date: May 9, 2018

(30) Foreign Application Priority Data
Nov. 9, 2015 (IN) 4269/MUM/2015

(51) Int. Cl. C07C 49/84; A61P 9/08

(52) U.S. Cl. CPC C07C 49/84; A61P 9/08

(57) ABSTRACT

Curcumin compositions are described for administration to healthy subjects, in need thereof, for improving cardiovascular flow. Curcumin compositions described herein can be administered to a subject in an effective dose of 250 mg to 1000 mg, corresponding to about 50 mg to 200 mg curcuminoids, for improvement of endothelial function and reducing risk of cardiovascular diseases. Compositions herein include curcumin either alone or curcumin along with at least one excipient to enhance absorption. Compositions herein improve endothelial function in healthy subjects having FMD value of 5-7% or higher and who are not diagnosed with cardiovascular disease. Compositions herein also improve cardiovascular endothelial function by reducing vascular resistance and enhancing nitric oxide generation in vascular endothelium. Curcumin compositions herein are safe for consumption, possesses enhanced absorption and can be employed for improvement of endothelial function and cardiovascular flow, when administered in an effective dose to a healthy subject, in need thereof.

### FIELD

Curcumin compositions described herein are administered to healthy subjects, in need thereof, for improvement in cardiovascular flow and reducing risk of cardiovascular diseases. More specifically curcumin compositions comprising curcuminoids as described herein can be administered to subjects in effective doses of 250 mg to 1000 mg, corresponding to about 50 mg to 200 mg of curcuminoids, for improvement in endothelial function and maintenance of healthy circulation, thus improving cardiovascular flow to reduce risk of cardiovascular diseases (CVD). The compositions include curcumin alone or include curcumin formulated with at least one excipient to form curcumin compositions having enhanced absorption. More particularly methods are described and include use of curcumin compositions, comprising curcuminoids, in healthy subjects identified with flow mediated dilation (FMD) value of about 5-7% or higher than 7% and/or people with potential risk of CVD based on their FMD value with known predisposing etiological factors. Predisposing etiological factors include refers to substance, event, characteristic or condition that contributes to the cardiometabolic disease's such as smoking, obesity, diabetes. Curcumin compositions described herein help to improve endothelial function in healthy subjects by regulating vascular tone, cellular proliferation, leukocyte adhesion, and platelet aggregation. Compositions herein help to reduce risk factors associated with cardiovascular diseases in healthy subjects having FMD value of about 5-7% or higher than 7% and who are not diagnosed with any acute or chronic illness and or endothelial dysfunction. This refers to a healthy subject with FMD value 5-7% who does not have measurable or significant endothelial dysfunction. Curcumin compositions herein also manage risk factors related to endothelial dysfunction such as hypertension, obesity, diabetes alone or in combination with one or more other risk factors related to cardiovascular flow, when administered to a healthy subject in need thereof, in an effective low and/or effective high dose. The compositions also improve endothelial function by reducing vascular resistance, assisting healthy circulation and vasodilation properties and enhancing nitric oxide generation in vascular endothelium, thus improving cardiovascular flow. Compositions herein are safe for consumption, possess enhanced bioavailability and can be employed for improvement of endothelial function and cardiovascular flow and reducing risk of associated conditions, when administered in an effective dose to a healthy subject, in need thereof.

### BACKGROUND

Significant lifestyle changes in the second half of the 20^{th} century have greatly contributed to the emerging epidemic of chronic diseases such as cardiovascular diseases (CVD). Currently, 15.3 million people are estimated to die from cardiovascular diseases every year; representing one-third of all global deaths from all causes. In the next two decades, the increasing burden of cardiovascular diseases will be borne mostly by developing countries.

Cardiovascular disease is a class of diseases that involve the heart or blood vessels. This includes multiple disorders such as angina, myocardial infarction (commonly known as a heart attack), stroke, hypertensive heart disease, rheumatic heart disease, cardiomyopathy, fibrillation, congenital, endocarditis, peripheral artery disease and venous thrombosis. All these conditions are prevalent in subjects wherein cardiovascular blood flow is affected due to endothelial dysfunction.

The majority of CVD is caused by risk factors that can be controlled, treated or modified, such as high blood pressure, cholesterol, overweight/obesity, tobacco use, poor diet, lack of physical activity, excessive alcohol consumption and diabetes. Nearly 34000 premature deaths of heart disease occur each year in the United States among nonsmokers. Hence timely identification, detection and treatment of the risk factors such as cardiac arterial diseases and improvement of cardiovascular blood flow is a critical step for avoiding the death rate from cardiovascular diseases.

Endothelial dysfunction is one of the very important early events to cardiovascular risk factors and precedes the development of cardiac artery disease and atherosclerosis with gross morphological signs and clinical symptoms. It is a systemic pathological state of the endothelium (the inner lining of blood vessels) and can be broadly defined as an imbalance between vasodilating and vasoconstricting substances produced by (or acting on) the endothelium. Normal functions of endothelial cells include mediation of coagulation, platelet adhesion, immune function and control of volume and electrolyte content of the intravascular and extravascular spaces. Endothelial dysfunction is a major pathophysiological mechanism that is caused by deficiency of Nitric Oxide (NO) and leads towards compromised cardiovascular flow, coronary artery disease, and other atherosclerotic diseases and it can also result from environmental factors, such as from smoking tobacco products and exposure to air pollution (Hua Cai et al 2000). Impaired endothelium-dependent vasodilation (endothelial dysfunction) in the coronary circulation of humans has profound prognostic implications in that it predicts adverse cardiovascular events and long-term outcomes. Hence it is important to assess endothelial dysfunction timely, even in healthy individuals so that risk of cardiovascular diseases can be predicted and corrective measures can be taken. Measurement of FMD is one of the ways to assess the endothelial function (Hadi et al 2005).

Another way of preventing cardiovascular diseases is consumption of the right diet, which is free of saturated fats and full of dietary fibers, fruits, vegetables, legumes, fish, wholegrain cereals and other essential nutrients. Thirty minutes of moderate physical activity every day may be sufficient to raise fitness of the heart and lungs which in turn may reduce the risk of future cardiovascular ailments. A longer duration and a higher activity level could provide even greater benefit to keep the disorders at bay.

Literature references describe evaluation of curcumin compositions in animal models and human beings for applications such as oxidative stress reduction and vascular system protection. Studies have also shown that curcumin possesses many biological activities including anti-inflammatory, anti-oxidant, and anti-microbial action (Maheshwari et al., Life Sciences 7%8:2081-2087%, 2006).

Boonla (Nitric Oxide. 2014 Nov. 15; 42:44-53) describes the beneficial role of curcumin in the prevention and treatment of hypertension. The study was carried out to investigate protective effect of curcumin on vascular remodeling and oxidative stress in 2K-1C hypertension-induced male rats model by treating them with curcumin at a dose of 50 or 100 mg/kg/day (or vehicle). After 6 weeks of treatment, curcumin ameliorated hemodynamic performance in rats (P<0.05), by reducing blood pressure, increasing hindlimb blood flow and decreasing hindlimb vascular resistance. Hemodynamic restoration was associated with a reduction in plasma angiotensin converting enzyme level.

Mangipudi et al (J. Phys. Pharm. Adv. 2013; 3(3): 85-93) investigated the antioxidant and vascular protective effect of curcumin on vascular endothelial dysfunction subsequent to high glucose stress in vitro. Thoracic aortic rings, obtained from male wistar rats were mounted in an organ bath and isometric contractions were recorded. Rings were preconstricted with phenylephrine, and endothelium dependent relaxation was observed by using acetylcholine and endothelium independent relaxation was observed by using sodium nitroprusside. Curcumin could alleviate the high glucose induced acute endothelium dependent vascular dysfunction in rat thoracic aortic rings.

Patent application US 20100021533 describes a synergistic mixture of curcumin along with at least 9 other plant products that is formulated and is capable of improving a person's wellbeing, lowering the risks of cardiovascular and/or Alzheimer's diseases and/or lowering blood sugar. The formulation may be utilized as a food or a drink or a supplement or a drug or a cosmetic or a hygienic product.

U.S. patent application 20130005824 relates to a method of treating a patient for ischemic tissue damage, in tissues such as skin, myocardium and central nervous system by administering a curcuminoid or a pharmaceutically active metabolite or analog thereof, intravenously at a dose within the range of 0.01 μg/kg-100 μg/kg. The study was mainly based on studies of burn injury progression and it was found that intravenous therapy with curcumin could impede burn injury progression.

U.S. patent application 20060228403 relates to an admixture containing either an admixture of individual compounds or individual compounds delivered simultaneously in individual dosages of the compounds. Such compounds include at least one omega-3 polyunsaturated fatty acid or an ester thereof, in an amount effective for enhancing vascular health and promoting a healthy cholesterol profile, curcumin in an amount specific for reducing endothelial inflammation and decreasing platelet aggregation, at least one B vitamin in an amount effective for lowering plasma homocysteine. The formulation was described to be administered for supporting cardiovascular health in young or old population.

Akazawa et al (Nutr Res. 2012 October; 32(10):795-799) describe a study wherein the effects of curcumin ingestion and aerobic exercise training were investigated on flow-mediated dilation as an indicator of endothelial function in postmenopausal women. FMD values were found to be increased significantly and equally in the curcumin and exercise groups. The study indicated that curcumin ingestion and aerobic exercise training can increase flow-mediated dilation in postmenopausal women.

However the volunteers selected for this study had 2-4% baseline FMD before intervention, which indicates that selected population was from risk population category. The differences in FMD change between the exercise and curcumin groups were not statistically significant. FMD values did not come to normal after intervention (>7% FMD healthy circulation)

### SUMMARY

Although the above literature relates to curcumin compositions for use as anti-inflammatory or for treating ischemic conditions, there is no discussion in the literature about curcumin compositions with enhanced absorption or about a method of administering these in a low dose to a healthy subject who are identified as ‘at risk’ of developing cardiovascular diseases based on measurement of FMD for assessment of endothelial dysfunction. The experiments described herein were carried out on healthy subjects having FMD values in the range of 5-7%. Curcumin compositions, including curcuminoids, as described herein increase FMD values in a healthy subject, improve cardiovascular flow and reduce risk of related cardiovascular diseases in healthy subjects, when administered in an effective dose of about 250 mg to 1000 mg. Curcumin compositions described herein exhibit enhanced absorption and bioavailability and hence low doses, corresponding to about 50 mg of curcumioids are sufficient for improving cardiovascular flow.

The applicant has carried out rigorous experimentation trials to demonstrate that curcumin compositions with enhanced bioavailability when administered to healthy subjects, in low as well as high doses of about 250 to 1000 mg, it improves endothelial function at both low as well as high doses, corresponding to about 50 to 200 mg of curcuminoids. Curcumin composition as described herein is administered to healthy subjects having FMD values of about 5-7% or higher than 7%, who are at risk of developing cardiovascular diseases, but not diagnosed with any cardiovascular ailments at the time of trial. This refers to healthy subjects who are medically fit and do not have cardiovascular diseases at the time of trial. The compositions are comprised of curcumin alone or curcumin formulated with at least one excipient to prepare curcumin compositions having enhanced absorption. The compositions may be in the form of curcumin extract or may be formulated using suitable excipients to prepare convenient dosage forms. As described herein, at least one excipient may be selected from the group of, but not limited to a hydrophilic carrier, a fat, an antioxidant, diluents, stabilizer, surfactant and the like or the combination thereof, which are acceptable for pharmaceutical or nutraceutical formulations.

In an embodiment, effective low dose stands for dose of 250 mg of curcumin composition which corresponds to 50 mg curcuminoids.

In an embodiment, effective high dose stands for dose of 1000 mg of curcumin composition which corresponds to 200 mg curcuminoids.

Curcumin compositions described herein surprisingly help to improve endothelial function and cardiovascular flow by increasing FMD values, enhancing endothelial nitric oxide synthase (eNOS), inhibiting endothelin 1 (ET1) and activating peroxisome proliferator-activated receptor γ (PPARγ), thus regulating vascular tone, cellular proliferation, leukocyte adhesion and platelet aggregation. This results into reduced vascular resistance and enhanced nitric oxide generation in vascular endothelium. Curcumin compositions herein also manage associated risk factors such as insulin resistance, atherosclerosis, pulmonary hypertension, inflammation and the like, when administered to a healthy subject in need thereof, in effective low and high doses. Curcumin compositions as described herein significantly improve endothelial dysfunction in apparently healthy subjects without established cardiovascular disease risk factors, even at a low effective dose, corresponding to about 50 mg of curcuminoids.

In an embodiment herein curcumin compositions comprising curcuminoids are provided, which when administered to a healthy subject in need thereof, are useful at a low as well as a high effective dose for improvement of endothelial function and cardiovascular flow in healthy as well as diseased subjects.

In an embodiment herein, curcumin composition comprised of curcumin alone or curcumin formulated with at least one excipient are provided. The compositions may be in the form of curcumin extract or may be formulated as convenient dosage forms using suitable excipients. Curcumin composition may contain at least one excipient selected from the group of, but not limited to a hydrophilic carrier, a fat, an antioxidant, diluents, stabilizer and surfactant or the combination thereof, which are acceptable to prepare pharmaceutical and nutraceutical formulations. These compositions include curcuminoids and exhibit enhanced absorption as well as superior bioavailability.

In an embodiment herein, curcumin compositions are provided such as by administration for management of cardiovascular diseases, when administered to a healthy subject at risk of developing such diseases.

In an embodiment herein, a method includes administering a curcumin composition to a healthy subject in a low dose such as at 200 mg which includes 50 mg curcuminoids for improvement of endothelial function. The healthy subject exhibits flow mediated dilation (FMD) values of at least 7%, but may or may not diagnosed with any cardiovascular disease or associated condition at the time of trial.

In an embodiment herein, a curcumin composition includes curcuminoids and is provided such as by administration, wherein the composition helps to improve endothelial function by regulating vascular tone, cellular proliferation, leukocyte adhesion, and platelet aggregation.

In an embodiment herein, curcumin compositions with enhanced absorption are provided such as by administration, wherein the compositions enhance eNOS, reduce ET1 and activate PPARy receptors and thus improve cardiovascular endothelial function by reducing vascular resistance and enhancing nitric oxide generation in vascular endothelium.

In an embodiment, curcumin compositions are provided such as by administration to manage associated risk factors such as insulin resistance, atherosclerosis, pulmonary hypertension, inflammation and the like, when administered to a healthy subject in need thereof, in an effective low or an effective high dose. The composition is safe for consumption, possess enhanced bioavailability and can be employed for improvement of endothelial function and cardiovascular flow, when administered to a healthy subject, in need thereof.

### BRIEF DESCRIPTION OF THE DRAWINGS

FIG. 1 shows a graph of the effect of 56 days of curcuminoids supplementation on FMD.

### DETAILED DESCRIPTION

Endothelial dysfunction is thought to be a key event in the development of atherosclerosis due to impaired cardiovascular flow and has been reported to predate clinically obvious vascular pathology by many years. It is associated with reduced anticoagulant properties as well as increased adhesion molecule expression, chemokine and other cytokine release, as well as reactive oxygen species production from the endothelium. This leads to inflammation and myofibroblast migration and proliferation inside the vessel all of which play important roles in the development of atherosclerosis, which is one of the cardiovascular disorders.

Endothelial dysfunction can be assessed by checking vasodilatation of an artery following an increase in luminal blood flow and internal-wall shear stress. Of all the current tests employed in the research setting, flow-mediated dilation is the most widely used in-vivo non-invasive test for assessing endothelial function. This technique measures endothelial function by inducing reactive hyperemia via temporary arterial occlusion and measuring the resultant relative increase in blood vessel diameter via ultrasound. FMD is widely believed to reflect endothelium-dependent and largely nitric oxide-mediated arterial function and has been used as a surrogate marker of vascular health. This technique has been used to compare groups of subjects and to evaluate the impact of interventions within individuals.

Curcumin compositions describe herein are provide and used for improvement of endothelial function and reduction in risk of cardiovascular disorders, thus managing cardiovascular disease. Methods described herein relate to administering curcumin compositions to a healthy subject, in need thereof, for management of cardiovascular diseases. Compositions described herein, improve FMD values in healthy subjects significantly above the baseline and decrease the risk of cardiovascular disorders, at both low and high doses of about 250 and 1000 mg, corresponding to about 50 to 200 mg of curcuminoids. Curcumin compositions described herein, may be administered in an effective amount, to a healthy subject in need thereof, for improving vascular tone, vascular function, insulin sensitivity and reducing risk of cardiovascular disorders, by improvement of cardiovascular flow.

The term ‘subject’ herein may relate to “a subject desiring improvement in endothelial function and cardiovascular flow such as a mammal, selected from a group of, but not limited to an animal, a human being who is healthy and may not be diagnosed with any cardiovascular disorder at the time of study. Such subjects may be at risk of developing cardiovascular disease condition because of some existing health disorders such as diabetes, septic shock, hyperlipidemia or may also result from environmental factors, such as from smoking tobacco products and exposure to air pollution. Group of subjects was tested for FMD to assess endothelial function and the subjects having about 5-7% FMD or higher than 7% were administered curcumin compositions at two different doses. In some circumstances, low FMD values, for example less than 7%, is considered as unhealthy FMD value as it indicates endothelial dysfunction; however the compositions of the invention may be administered to healthy and/or diseased subjects for improvement in FMD.

The term ‘effective dose’ relates to the dose of curcumin compositions which is administered to healthy subjects on a daily basis (e.g. over at or about 56 days or at or about 8 weeks) and found to have desired effect in terms of increase in FMD values. The effective dose as described herein corresponds to 250 mg to 1000 mg of a curcumin composition, which corresponds to about 50 mg to 250 mg of curcuminoids comprised in these curcumin compositions. It should be noted that sometimes in general terms the term curcuminoids and curcumin may be used interchangeably. The study subjects were administered with this dose for 8 weeks of supplementation. In an embodiment, the daily dose can be for a duration of at or about 30 days to at or about 180 days. Other ranges falling within the dose range of 250 to 1000 mg of curcumin composition also fall within the scope of this term and are considered as an ‘effective dose’. The effective dose is considered in terms of corresponding content of curcuminoids present in the curcumin composition. The term ‘low dose’ as used herein is relative for example with respect to other regimen given to another set of subjects in the form of dose of 250 mg curcumin composition which corresponds to 50 mg curcuminoids.

The term ‘improvement in cardiovascular risk’ relates to improvement of endothelial function by improving FMD values and thus enhancing blood flow through vascular system. This may bring out protection of healthy subjects from risk of developing cardiovascular diseases at later phase of life, by identifying the risk at earlier stage with the help of non-invasive clinical investigations. Curcumin compositions herein are administered at particular doses to healthy subjects identified with unhealthy FMD values of at least or less than 7% in some cases (e.g. about 5-7% or higher than 7%), which can be considered as risk of developing cardiovascular diseases at later phase of life, because of compromised endothelial function. The compositions also may be administered to subjects already diagnosed with some of the risk factor and/or disease and prescribed with the medicine along with diet and exercise. Curcumin compositions as described herein can act as nutraceutical or therapeutic adjuvant to improve endothelial function significantly in healthy subjects and thus reduce risk of cardiovascular disorders, by improving cardiovascular flow.

Curcumin compositions herein are comprised of the lipophilic active curcumin [1,7%-bis(4-hydroxy-3-methoxyphenyl)-1,6-heptadien-3,5-dione] which is a polyphenol derivative derived from the spice turmeric. Commercial curcumin can sometimes contain approximately, 7% diferuloylmethane, 17% demethoxycurcumin, 6% bisdemethoxycurcumin and tetrahydrocurcumin. Curcumin is a major active ingredient of Curcuma Longa. Curcuma longa (turmeric) is a well-known indigenous herbal medicine. It is known for its diverse biological actions and pharmacological activities including anti-inflammatory, antioxidant, antiproliferative, antimicrobial, anticarcinogenic and antiangiogenic properties. It is to be appreciated that the term “curcumin” can be interpreted to be within the scope of the term curcuminoids, which can in general include components curcumin, methoxy curcumin, demethoxy curcumin, bisdemethoxy curcumin and tetrahydrocurcumin. Commercial products which may be referred to as “curcumin” may have these three components, along with other components belonging to the class curcuminoids.

It is also to be appreciated that diarylheptanoid are considered a class of compounds to which curcuminoids (e.g. curcumin) belongs. Other similar Diarylheptanoids, such as those that may be obtained from ginger may possess similar properties as curcuminoids (e.g. curcumin). It will be appreciated that while curcuminoids (e.g. curcumin) may be described in detail herein, it will be appreciated that other diarylheptanoids may have similar or the same biological properties and effects, and which may be included in such compositions as described herein and may be used in the methods of treatment as described herein.

The beneficial effects of curcumin have been well known. However, there are many problems associated with the bioavailability of curcumin when delivered in the oral form. Major portion of ingested curcumin is excreted through the feces unmetabolized and the small portion that gets absorbed is converted into other metabolites and excreted. Curcumin does not easily penetrate the gastrointestinal tract and is subject to liver and other intestinal enzymes. Owing to these enzymes, the curcumin within the body is rapidly metabolised thus reducing its bioavailability in the body. The small amount of curcumin that enters the bloodstream is rapidly metabolized by the liver and kidney. Therefore, although curcumin is highly lipophilic (and so easily crosses the blood brain harrier), only very small amounts of orally administered curcumin are detected in the serum and in the brain tissue.

Cytochrome P450 is a phase I metabolizing isoenzyme which is required for metabolizing toxic chemicals such as heterocyclic amines to induce DNA adduct formation leading to carcinogenesis. Curcumin when ingested in the body enters the gastrointestinal tract and is found to inhibit Cytochrome P450. There have been studies carried out to increase the bioavailability of curcumin when used along with piperine. The compositions described herein are seen to enhance the bioavailability through enhanced absorption without the presence of any additional bioenhancer.

Curcumin compositions described herein are comprised of either curcumin alone or in combination with at least one or more pharmaceutically and/or nutraceutically acceptable excipients to increase absorption of curcumin. More preferably, curcumin composition is formulated using excipients selected from the group of, but not limited to a hydrophilic carrier, cellulosic polymers, solubilizers, stabilizers, emulsifiers, fats, diluents, binder, antioxidants, and the like or the combinations thereof. When curcumin is combined with at least one pharmaceutically or nutraceutically acceptable excipient such as a hydrophilic carrier and formulated as a solid dispersion of curcumin by adding other excipients, the bioavailability of such optimized curcumin formulation is enhanced.

Curcumin compositions, as described herein are also comprised of curcumin and at least one excipient such as hydrophilic carrier, which can be formulated as spray dried free flowing soluble granules.

In some embodiments, a process for the preparation of a curcumin composition suitable for enhancing endothelial function comprises; dissolving curcumin, at least one excipient such as hydrophilic carrier and adding other excipients such as a fat and an antioxidant, in a solvent to form a homogenous mass; warming the resultant mass at a temperature ranging from 25° C. to 60° C. for a period of about 4 to 8 hours to obtain a dry wet mass; removing the solvent by evaporation to form a dry mass; and pulverizing the dry mass to form a fine powder.

In some embodiments, the process for preparation of soluble granules and/or beadlets of curcumin is described, wherein curcumin is dispersed in solid hydrophilic carrier along with at least one more pharmaceutical or nutraceutical excipient in suitable organic solvent, which is safe for human consumption and spray dried to get the product. These granules can be filled in sachets as one of the convenient way for administration or can be suspended in suitable oil medium with stirring and followed by milling to get uniform suspension.

In some embodiments, curcumin used in the step (i) can be commercially available in the form of an extract of turmeric rich in curcuminoids.

In some embodiments, the solid hydrophilic carrier employed in preparation of curcumin compositions is selected from the group such as, but not limited to, cellulose derivatives, polyacrylates, polyethylene glycols, povidones, starch and starch derivatives, gums, sugars, and the like. The compositions may/may not be free of povidone as hydrophilic carrier.

In some embodiments, the hydrophilic carrier used in the step (i) can be selected from soluble starch, hydroxy propyl methyl cellulose, sodium carboxy methyl cellulose, polyvinyl pyrrolidone, polyethylene glycols 200-20000, glycerol, sorbitol, mannitol, glucose, sugar, and mixtures thereof. The quantity of hydrophilic carrier added may range between 10-90%.

In some embodiments, the fat used in the step (i) may be selected from milk fat, medium chain triglycerides, long chain triglycerides, hydrogenated vegetable oils, esters of fatty acids, hydrocarbons such as terpenes and mixtures thereof. The quantity of fat used may range from 1-25%.

In some embodiments, at least one more excipient employed in the composition may be selected from, but not limited to diluents, binder, surfactant, solubilizer, antioxidant, solvent and the like or the combinations thereof. The antioxidants used in step (i) can be selected from natural tocopherols, ascorbyl palmitate, rosemary extract, epigallocatechin gallate, catechins, ascorbic acid, and mixtures thereof. The amount of antioxidant used may range between 1-10%.

In some embodiments, antioxidant such as ascorbyl palmitate may also perform function of fatty excipient in the composition, thus acting in dual role of antioxidant and fatty excipient.

The solvent used for dissolving in the step (i) may be selected from isopropyl alcohol, acetone, methanol, alcohol, and mixtures thereof. The temperature maintained for obtaining an homogenous mass may range from ambient to 7%0° C.; preferably 25° C. to 60° C.

The removal of solvent in step (ii) can be performed in vacuum distillation or evaporation technique, or by spray drying technique. The resultant dry mass is pulverized by using, for example, mortar and pestle, mixer-grinder, multi-mill, ball mill, jet mill and the like.

The compositions may comprise curcumin, a hydrophilic carrier, a fat and at least one more excipient such as an antioxidant. The antioxidant along with curcumin can inhibit the Cytochrome P450. On the other hand, the presence of fat coating on the composition can prevent the composition from attack by liver microsomal or other intestinal enzymes as these enzymes attack only aqueous compounds. Thus, the antioxidant and the fat can enhance the bioavailability of curcumin.

Curcumin compositions described herein exhibit enhanced bioavailability and the compositions can be available in orally administrable solid, semisolid, liquid forms, selected from, but not limited to dosages such as, powders, granules, pellets, beadlets, caplets, tablets, capsules, soft gel capsules, solution, emulsions, suspensions, oil suspensions, dispersions and the like.

In some embodiments, curcumin compositions described herein are evaluated for their effects on FMD and endothelial function of healthy subjects to check for use in management of cardiovascular diseases. The evaluation is done in healthy subjects identified with FMD value of at least 7%.

Applicant has evaluated curcumin compositions for their effect on vascular endothelial function in an effective dose in a healthy subject in terms of flow mediated dilation as non-invasive technique for endothelial function. In an embodiment, curcumin compositions herein, improve FMD values by at or about 3% in healthy subjects having FMD at or about 5-7% or at least 7%. In an embodiment, curcumin compositions herein, improve FMD values by at or about 3.3% in healthy subjects having FMD at or about 5-7% or at least 7%, thus reducing risk of cardiovascular disorders by about 60%, when administered at low dose of 250 mg curcumin composition corresponding to about 50 mg curcuminoids, which is nowhere reported in the prior art.

In some embodiments, curcumin compositions described herein, improve endothelial function in healthy subjects having FMD as low as about 5-7%, at low as well as a higher dose, corresponding to 50 mg and 200 mg curcuminoids.

Nutrigenomics study indicates that curcumin compositions decrease ET1 (endothelin 1), enhance eNOS (endothelial nitric oxide synthetase) and activate PPARγ receptors, which are essential for healthy cardiovascular system. eNOS is primarily responsible for the generation of NO (Nitric Oxide) in the endothelium, a monolayer of flat cells lining the interior surface of blood vessels, at the interface between circulating blood in the lumen and the remainder of the vessel wall. NO produced by eNOS in the vascular endothelium plays crucial roles in regulating vascular tone, cellular proliferation, leukocyte adhesion, and platelet aggregation. Therefore, a functional eNOS is essential for a healthy cardiovascular system.

Endothelin 1, also known as preproendothelin-1 (PPET1), is a protein that in humans is encoded by the EDN1 gene. The protein encoded by this gene is proteolytically processed to release a secreted peptide termed endothelin 1. This peptide is a potent vasoconstrictor and is produced by vascular endothelial cells. Endothelin 1 is one of three isoforms of human endothelin (ET-1). PPAR-γ is highly expressed in adipose tissue and plays a crucial role in adipocyte differentiation. It is also expressed in a variety of other tissue and cell types, where it plays key roles in the regulation of metabolism and inflammation.

PPAR gamma activation helps to decrease insulin resistance and improves insulin sensitivity and reduces risk of CVD, reduces inflammation and potential treatment for cancer, adipocyte differentiation and reducing inflammation will reduce the risk of obesity and related cardiometabolic ailments such as diabetes, atherosclerosis and inflammatory diseases.

In some embodiments, FMD is measured in healthy subjects who are not diagnosed with any cardiovascular disorder at the time of study. Brachial artery diameter is measured during three conditions; baseline (after at least 10 min supine rest), during reactive hyperaemia (induced by inflation to 250 mm Hg and then deflation of a sphygmomanometer cuff around the forearm) and finally after the administration of sublingual nitroglycerin. A linear array, high resolution ultrasound transducer is used to provide B-mode images of the target vessel, proximal to the forearm cuff.

In some embodiments, healthy subjects with FMD value of about 5-7% or higher than 7% were selected for the study and curcumin composition corresponding to 50 mg and 200 mg curcuminoids were administered as supplements over 8 weeks duration. Baseline FMD and end point FMD were checked in these subjects. Healthy subjects identified with FMD less than 7% exhibited significant improvement in FMD at both the doses.

Curcumin compositions as described herein, when administered to healthy subjects at low dose, having FMD value of about 7%, increased FMD significantly by 3 to 4% and reduced cardiovascular risk by 25 to 65%. Thus the compositions here demonstrated protection of healthy subjects from risk of developing cardiovascular diseases at later phase of life, by identifying the risk at earlier stage with the help of non-invasive clinical investigations. Curcumin compositions herein are also administered at particular doses to healthy subjects identified with unhealthy FMD values of less than 7%, which can be considered as risk of developing cardiovascular diseases at later phase of life, because of compromised endothelial function. Curcumin compositions as described herein improve endothelial function significantly in healthy subjects and thus reduce risk of cardiovascular disorders, thus managing cardiovascular diseases.

Curcumin compositions described herein improve endothelial function by enhancing eNOS, inhibiting ET1 and activating PPARγ receptors, thus regulating vascular tone. This results in reduced vascular resistance and enhanced nitric oxide generation in vascular endothelium. Curcumin compositions as described herein significantly improve endothelial dysfunction and cardiovascular flow in healthy subjects who are at risk of developing cardiovascular diseases, thus managing risk conditions even at low doses.

While the compositions and methods herein have been described in terms of specific illustrative embodiments, any modifications and equivalents that would be apparent to those skilled in the art are intended to be included within the scope of the compositions and methods herein. The details of the compositions and methods herein, its objects, and advantages are explained hereunder in greater detail in relation to non-limiting exemplary illustrations.

### Examples

A. In—Vivo Study

This study was to evaluate the effects of 8 weeks of curcumin composition supplementation with two different doses on flow mediated dilation (FMD) of the brachial artery in comparison to control. The experiments herein were carried out on healthy subjects having FMD values in the range of 5-7%.

Study Design: Double blind placebo randomized controlled study using 63 healthy volunteers over duration of 8 weeks. Experiments show significant results on 8 weeks supplementation

Eligibility criteria for selection of healthy volunteers: non-smoking healthy men and women aged 19 to 29 years with no musculoskeletal, medical, or metabolic contraindications to exercise. Interventional product: Placebo, 250 mg Curcumin composition (corresponding 50 mg Curcuminoids) and 1000 mg Curcumin composition (corresponding to 200 mg Curcuminoids).

Study time points: Baseline and Final visit Study End points: Endothelial function was assessed by flow-mediated dilation (FMD).

Prior to experimental testing, subjects completed preliminary medical history, exercise and demographic questionnaires along with height and body weight determination. Subsequently, endothelial function (flow-mediated dilation, FMD) was assessed followed by maximal aerobic capacity (VO₂ₘₐₓ) Participants were then matched according to body mass and randomly assigned to ingest, in a double blind manner, capsicum composition comprising either 50 mg curcuminoids (LOW) or 200 mg curcuminoids (HIGH), or placebo (P). The day following baseline testing, participants were asked to ingest one equal dose capsule with breakfast, lunch and dinner; three capsules per day in total.

Participants were advised to maintain their current diet and exercise program for the duration of the 8-week supplementation period. At the conclusion of the 8-week supplementation (56 days), all experimental testing procedures were repeated.

Determination of Maximal Aerobic Capacity (VO₂ₘₐₓ)

Maximal aerobic capacity was determined using an incremental treadmill test to exhaustion. Throughout the test, respiratory gas exchange was measured using an open-circuit gas analysis system and heart rate was monitored using a telemetry system (Polar Electro E600, Polar Electro Inc, Lake Success, New York). This test was performed to ensure that regular exercise activity results in likely clinical improvement in endothelial function.

Determination of Flow-Mediated Dilation (FMD)

Endothelial function was assessed using the standard flow-mediated dilation (FMD) test (Stoner & Sabatier, 2012 J Atheroscler Thromb, 19(5), 407-421).

Participants were examined in a sound-isolated, temperature-controlled room following an overnight fast (>10 h) for baseline testing.

Post-supplementation testing was performed on day 56 for all female subjects coincident with the baseline testing cycle stage. Brightness mode ultrasound measurements were made using an Acuson Aspen Ultrasound system (Mountain View, Calif.). A standard adult blood pressure cuff was wrapped around the right forearm approximately 10 cm distal to the antecubital space. Following a quiet rest period in the supine anatomical position of at least 10 min, the right brachial artery diameter was scanned. Following collection of baseline images (3×8 second clips), the blood pressure cuff was inflated to 50 mm Hg above resting systolic blood pressure for 5 minutes. Imaging of the vessel diameter resumed 30 seconds prior to cuff deflation and continued for 3 minutes post deflation (7×30 second clips). Baseline and post-deflation vessel diameters were analyzed using semi-automated brachial analyzer software (Medical Imaging Applications, LLC; Coralville, Iowa).

FMD was calculated as (maximum diameter−baseline diameter)/baseline diameter×100, where the maximum diameter represents the maximum diameter post 5 minutes distal ischemia.

Results

Safety data obtained from venous blood sampling (complete blood count and metabolic panel) were within normal range and no adverse events were observed during 8 week supplementation.

Maximal Aerobic Capacity VO₂ₘₐₓ

A small most likely trivial improvement in VO2max was observed in all treatment groups indicating that the exercise performed over the 8-week intervention was not sufficient to induce significant adaptations such as improved endothelial function.

Flow Mediated Dilation

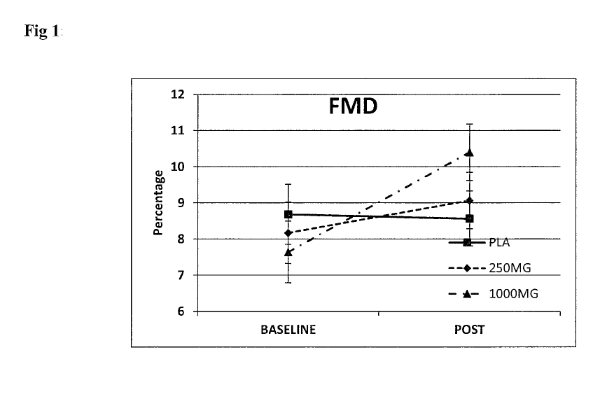

The effect of 56 days of curcuminoids supplementation on FMD is shown in FIG. 1. The main finding of this study is a clinically substantial 3.0% increase in FMD following 8 weeks high dose (200 mg) curcuminoids supplementation, and 1.7% improvement following low dose (50 mg) supplementation. Considering FMD is the standard test of endothelial function, these findings provide preliminary evidence to support the notion that chronic curcuminoids supplementation may decrease the risk of CVD in persons who are apparently healthy.

TABLE 1
Baseline Characteristics of Healthy Volunteers
Baseline Characteristics

| Details | PLA, (n = 21) | 250 mg, (n = 20) | 1000 mg (n = 22) |
|---|---|---|---|
| Age (y) | 21 ± 2 | 21 ± 2 | 22 ± 2 |
| BMI, Kg/mg² | 23.8 ± 2.6 | 23.8 ± 3.5 | 23.5 ± 3.1 |
| VO₂ₘₐₓ | 41.6 ± 6.0 | 43.1 ± 7.1 | 43.6 ± 7.3 |
| (ml · kg · min⁻¹) | | | |
| FMD, % | 9.16 ± 4.81 | 8.2 ± 3.3 | 7.6 ± 3.2 |

There was no significant difference in baseline characteristics of healthy volunteers.

TABLE 2
Effect of curcumin supplementation on FMD values

| | | 250 mg Curcumin | 1000 mg Curcumin |
|---|---|---|---|
| Visits | Placebo | composition | composition |
| Baseline | 9.16 ± 4.81 | 8.2 ± 3.3 | 7.6 ± 3.2 |
| Final Visit | 8.8 ± 3.9 | 9.1 ± 2.7 | 10.4 ± 3.4* |

*p < 0.005

The study demonstrated a significant increase in % FMD over placebo and baseline only in the high dose (1000 mg Curcumin composition corresponding to 200 mg curcuminoids).

The effect at the high dose may be as a result of a lower FMD (%) in the group at baseline.

Significant increase in FMD was observed at final visit only with dose of 1000 mg Curcumin composition. A slight increase in FMD was observed in 250 mg dose of curcumin composition. There was a decrease in FMD in placebo group at final visit.

1. A curcumin composition comprising at least 50 mg curcuminoids, for use in improving cardiovascular flow when administered in an effective dose to a healthy subject in need thereof.

2. The composition of claim 1, which improves cardiovascular flow and reduces risk of cardiovascular diseases in healthy subjects, having FMD (Flow mediated dilation) values of 5-7% or higher.

3. The composition of claim 2, which improves cardiovascular flow in healthy subjects having FMD value of 5-7% or higher, who are not diagnosed with any cardiovascular disease.

4. The composition of claim 3, which improves cardiovascular flow by exhibiting action on endothelial function, when administered in effective daily dose to healthy subjects in need thereof.

5. The composition of claim 4, which improves endothelial function by enhancing flow mediated dilation when administered in effective daily dose to healthy subjects in need thereof.

6. The composition of claim 5, which improves cardiovascular flow through increasing FMD values by 1 to 4%, when administered in effective daily dose to healthy subjects in need thereof.

7. The composition of claim 1, which is administered in effective doses of 250 mg to 1000 mg, so that it provides about 50 mg to 200 mg of curcuminoids to healthy subjects in need thereof for improving cardiovascular flow by increasing FMD values.

8. The composition of claim 1, which is administered in doses of 500 mg to 800 mg, so that it provides about 100 mg to 160 mg of curcuminoids to healthy subjects in need thereof for improvement in cardiovascular flow and reduction in risk of cardiovascular diseases.

9. The composition of claim 1, which is comprised of at least one excipient, which when administered in effective daily dose to healthy subjects in need thereof, enhances FMD values and reduces risk of cardiovascular diseases.

10. The composition of claim 9, which is comprised of at least one excipient such as hydrophilic carrier and exhibits enhanced absorption, improves endothelial function and reduces risk of cardiovascular diseases, when administered in effective daily dose to healthy subjects in need thereof.
